(19) United States
(12) Patent Application Publication
(10) Pub. No.: US 2026/0234704 A1
(43) Pub. Date: Aug. 13, 2026

(54) A METHOD FOR IN-SITU PROFILING OF SINGLE NUCLEOTIDE VARIATIONS IN AN RNA SAMPLE AND A KIT FOR USE IN IN-SITU PROFILING OF SINGLE NUCLEOTIDE VARIATIONS IN AN RNA SAMPLE

(71) Applicant: VOXLBIO AB, Bandhagen (SE)

(72) Inventors: Mats NILSSON BERNITZ, Drottningholm (SE); Marco GRILLO, Sundbyberg (SE); Hao Zhe LEE, Solna (SE)

(21) Appl. No.: 19/147,843

(22) PCT Filed: Feb. 2, 2024
(86) PCT No.: PCT/SE2024/050091

§ 371 Date: Jul. 14, 2025

(30) Foreign Application Priority Data
Feb. 2, 2023 (SE) 2350098-6

(51) Int. Cl. C12Q 1/6841; C12Q 1/6827

(52) U.S. Cl. CPC C12Q 1/6841; C12Q 1/6827

(57) ABSTRACT

The disclosure relates to a method for in-situ profiling of single nucleotide variations in an RNA sample, comprising the steps of: (a) contacting the RNA sample with a plurality of chimeric padlock probes comprising an modified base at the 3′ end portion; (b) adding an RNA ligase under conditions and with reagents allowing ligation of the padlock probes with a single nucleotide specificity, in order to generate a circularized padlock oligonucleotide for padlock probes comprising a modified base that is complementary with the corresponding position of the RNA sample; (c) amplifying the circularized padlock oligonucleotide under conditions and with reagents allowing rolling circle amplification, thereby generating an amplified circularized padlock oligonucleotide; (d) detecting the amplified circularized padlock oligonucleotide, thereby obtaining a profile of single nucleotides in the RNA sample. The disclosure further relates to a kit for use in in-situ profiling of single nucleotide variations in an RNA sample.

### TECHNICAL FIELD

The present disclosure relates to a method for in-situ profiling of single nucleotide variations in an RNA sample and a kit for use in in-situ profiling of single nucleotide variations in an RNA sample.

### BACKGROUND ART

mRNA detection using RNA-templated ligation of padlock probes followed by rolling circle amplification is a recently established method for in-situ mRNA analysis. Currently, this approach is based on two alternative but similar method variants: 1) DNA padlock probes are ligated onto the target mRNA using SplintR ligase (https://doi.org/10.1371/journal.pbio.3000675), or 2) chimeric RNA probes (DNA probes with a 3′ terminal ribose) are ligated onto the target mRNA using T4 RNA Ligase 2 (T4 Rnl2) (doi: 10.1261/rna.066753.118). Both ligases, however, display a high mismatch tolerance (doi: 10.1261/rna.066753.118). This comes with two major hindrances: on one hand, padlock probes cannot be used to discriminate SNPs or even small deletions directly on the target mRNAs. On the other hand, RNA-templated ligation of padlock probes fails to discriminate between the RNAs of closely related genes (i.e. recent gene duplications whose sequence has not dramatically diverged).

Krzywkowski et al. (RNA, 2019 January; 25 (1): 82-89, “Chimeric padlock and iLock probes for increased efficiency of targeted RNA detection”) investigates the use of DNA ligases on RNA substrates using padlock technology. However, RNA detection sensitivity was found to be limited.

Thus, the state of the art-approach to detect single nucleotide variations on mRNA currently requires one to first perform reverse transcription to generate cDNA from mRNA in-situ, and posteriorly detect the single nucleotide variation on the generated cDNA using DNA padlock probes with a very accurate DNA ligase (Tth). However, cDNA synthesis in situ is inefficient and creates a detection bottleneck, which results in low detection efficiency of single nucleotide variation events (https://dx.doi.org/10.18632% 2Foncotarget.1527).

Hence, currently there is no adequate method and/or reliable approach to be able to profile single nucleotide polymorphisms or variations (SNP/SNV) directly on mRNA in biological tissues in situ. The low detection efficiency of the cDNA approach is often unable to produce good quality data, especially when detecting poorly abundant mRNAs. In addition, the cDNA-based approach for SNP detection has a known low performance in FFPE tissue sections, presumably due to the fragmented mRNA content. The state of the art-approach to profiling SNP efficiently today employs the use of single-cell RNA sequencing (scRNA-seq) approaches. Although efficient, scRNA-seq approaches require the biological tissue to be dissociated in the process, resulting in the loss of spatial information.

Spatial information is important as biology inherently is heterogeneous and without the spatial information of cells researchers lose the ability to infer cell-cell interactions within the biological tissue itself. Such spatial information of SNPs is especially important in immuno-oncology research where researchers attempt to further their understanding of diseases such as cancer.

The cDNA-based In Situ Sequencing (ISS) approach seeks to address the spatial aspect of SNP profiling in situ, but due to the inefficiency of cDNA synthesis from mRNAs and post fixation, the detection efficiency of the approach is low and processing FFPE sections where RNA is known to be fragmented, is extremely challenging (Gyllborg et al., “Hybridization-based in situ sequencing (HybISS) for spatially resolved transcriptomics in human and mouse brain tissue”, Nucleic Acids Research, Volume 48, Issue 19, 4 Nov. 2020, Page e112, https://doi.org/10.1093/nar/gkaa792).WO2022087273 discloses methods for spatial analysis using padlock probe technology and subsequent rolling circle amplification, thereby facilitating identification of alternative splicing events, translocation events and mutations that change the hybridization rate of one or both probe oligonucleotides. Among a plurality of examples, template dependent ligation of oligonucleotides hybridizing to proximal target regions of an analyte is discussed, including, among others, the recently characterized Archaeal RNA ligase from Thermoccocus kodakarensis (KOD Ligase).

Also, WO2019068880 discloses a method for detecting target nucleic acid sequences in a target nucleic acid molecule in a sample. The use of various enzymes is discussed, including SplintR ligase and T4 RNA ligase II as ligases and Phi29 DNA polymerase as a polymerase. Also, the use of padlock probes with a flap at the 5′ end of the probe is disclosed. Further, WO2022256422 discloses asymmetric padlock probes (PLP) used for sample analysis of SNPs and point mutations.

Thus, the disclosures of the prior art do neither solve the problems of analyzing and identifying variations and mutations in RNA molecules in a reliable and efficient way, nor suggest adequate and effective solutions. Therefore, there is a need for improved methods and approaches for being able to profile variations in RNA molecules, especially for in situ applications.

### SUMMARY

It is an object of the present disclosure to mitigate, alleviate or eliminate one or more of the above-identified deficiencies and disadvantages in the prior art and solve at least one of the above-mentioned problems.

The present inventors have developed an approach using chimeric padlock probes with a single-nucleotide specific ligase that probes the SNP on mRNA directly, and is able to preserve single nucleotide specificity detection, addressing the issue of spatial resolution and also provide increased detection sensitivity and as such, is compatible with difficult FFPE tissue sections as the padlock probes (PLPs) are better able to target fragmented mRNA molecules as compared to the cDNA approach.

Hence, according to a first aspect there is provided a method for in situ profiling of single nucleotide variations in an RNA sample, comprising the steps of:
(a) contacting the RNA sample with a plurality of chimeric padlock probes, comprising a modified base at the 3′ end portion, wherein the modified base is chosen from C, A, G and U/T under conditions and with reagents allowing hybridization;
(b) adding an RNA ligase under conditions and with reagents allowing ligation of the padlock probes with a single nucleotide specificity, in order to generate a circularized padlock oligonucleotide for padlock probes comprising a modified base that is complementary with the corresponding position of the RNA sample;
(c) amplifying the circularized padlock oligonucleotide under conditions and with reagents allowing rolling circle amplification, thereby generating an amplified circularized padlock oligonucleotide;
(d) detecting the amplified circularized padlock oligonucleotide, thereby obtaining a profile of single nucleotides in the RNA sample.

Thus, the invention uses chimeric padlock probes with a suitable modified base on the 3′ end, together with a SNP-accurate ligase that is able to ligate padlock probes with single-nucleotide specificity, before performing rolling circle amplification, in order to profile single nucleotide variation (SNV) in situ.

Whether an RNA ligase exhibits “single-nucleotide specificity” could be determined and/or measured by comparing the specificity with a threshold and/or a reference value. E.g. the threshold and/or reference value could be a comparison of specific and non-specific signal, wherein e.g. the specific signal could be an order of magnitude greater than the non-specific signal to be sufficiently specific and providing a sufficiently strong signal. Alternatively, the current “state of the art” in-situ profiling is via a cDNA approach, which could be used as a benchmark (reference) value.

Hence, the inventors developed an approach using chimeric padlock probes with a ligase that probes the SNP on mRNA directly. This makes it possible to perform single nucleotide detection, in a spatially resolved manner, down to the level of subcellular resolution, and also provide increased detection sensitivity and as such, is compatible with difficult FFPE tissue sections as the padlock probes (PLPs) are better able to target fragmented mRNA molecules as compared to the cDNA approach. Thus, by a clever choice of ligase and padlock probe design, a specificity allowing discrimination and/or profiling of single-nucleotide variations is obtained.

According to some embodiments, the RNA sample comprises at least one nucleotide position to be profiled, flanked by nucleotide stretches of known identity at the 3′ and the 5′ side of the at least one nucleotide position to be profiled.

According to some embodiments, the RNA sample is an mRNA sample, or a rRNA sample, or a micro-RNA, or a non-coding RNA.

According to some embodiments, the plurality of chimeric padlock probes each have a first end and a second end, which first and second ends are designed to hybridize to the RNA sample to the nucleotide stretches of known identity at the 3′ and 5′ sides of the at least one nucleotide position to be profiled, respectively, so that the terminal base at the 3′ end, or any of the two bases next to the terminal base at the 3′ end, is positioned at the nucleotide position to be profiled in the RNA sample.

By “terminal base at the 3′ end” is typically meant the final base at the 3′ end.

According to some embodiments, the padlock probes having bases at the 3′ end that is complementary with the nucleotide in the nucleotide position to be profiled of the RNA sample are ligated in step (c), and wherein padlock probes having at least one base at the 3′ end that is not complementary with the nucleotide in the nucleotide position to be profiled of the RNA sample are not ligated in step (c).

According to some embodiments, the RNA ligase is chosen from the group comprising: (i) a KOD RNA ligase, chosen from KOD1Rnl from Thermococcus kodarensis, which ligase (not commercially available as of this date) has shown unexpectedly reliable results, (ii) PBCV-1 DNA Ligase/Chlorella virus DNA Ligase, (iii) engineered ligases from the PBCV-1 family, and (iv) engineered ligases from the family of archaeon, Thermococcous kodarensis.

The synthesis and characterization of this enzyme is disclosed by Zhang et al. (RNA Biol. 2017; 14 (1): 36-44, “Archaeal RNA ligase from Thermoccocus kodakarensis for template dependent ligation”), wherein the template dependent and thermostable properties, as well as potential in vitro applications are discussed.

According to some embodiments, the detection in step (e) is by means of luminescence, such as fluorescence, and/or by means of sequencing, such as Sequencing by Hybridization, Sequencing by Ligation, SOLID sequencing or Sequencing by Synthesis.

Hereby, profiling of single nucleotide variations in the RNA sample is readily obtained.

According to some embodiments, detection oligonucleotides are added to the amplified circularized padlock oligonucleotides, wherein the detection oligonucleotides are designed to bind directly to the amplified circularized padlock oligonucleotides in an RNA base specific manner, or to a bridging oligonucleotide that is designed to bind to the amplified circularized padlock oligonucleotides in an RNA base specific manner, so that the resulting luminescent signal allows profiling of the single nucleotide variation in the RNA sample.

According to some embodiments, the profile of the amplified circularized padlock oligonucleotides is distinguished by fluorescent readout by introducing different barcodes in the backbone sequence of the padlock probes for the competing PLP probes.

Hereby, the resolution and quality of the readout signal is further improved.

According to some embodiments, more than one single nucleotide variation positioned on the same RNA sample molecule are profiled. Hereby, multiple SNVs can be profiled in the same experiment. In order to profile multiple SNVs, chimeric padlock probes designed for each SNV to be profiled must be provided.

According to some embodiments, more than one single nucleotide variation positioned on different RNA sample molecules are profiled. Hence, multiple SNVs/mutations can be profiled across multiple transcript targets in one single experiment. Chimeric padlock probes designed for each SNV to be profiled must be provided.

According to some embodiments, the terminal base of the 3′ end of the chimeric padlock probes is chosen from (i) an RNA base, (ii) a 2′-O-methoxy-ethyl base, (iii) a 2′-O-methyl RNA base, (iv) a 2′-fluoro base, (v) a DNA base or (vi) an LNA base.

An “RNA base” is typically a normal RNA base, i.e. a non-modified A, G, C or U, whereas a “DNA base” typically refers to a norma DNA base, i.e. a non-modified A. G. C or T.

Hence, using modification at or close to the 3′ terminal end of the padlock probe may improve the specificity of the ligase and save costs, depending on choice of modified base in combination with choice of ligase.

According to some embodiments, the modified base at the 3′ end portion is positioned within at least two bases from the 3′ terminal base.

By “the 3′ end portion” is meant the about 2-3 final bases that are positioned at the 3′ end of the padlock probe,

According to some embodiments, the remaining bases of the 3′ end portion are non-modified DNA bases.

According to some embodiments, the padlock probe has any of the following designs:
(i) the 3′ terminal end base is a modified base and is positioned at the nucleotide position to be profiled of the RNA sample;
(ii) the 3′ terminal end base is a modified base, and the base next to the 3′ terminal end base is positioned at the nucleotide position to be profiled of the RNA sample; or
(iii) the base next to the 3′ terminal end base is a modified base and is positioned at the nucleotide position to be profiled of the RNA sample.

According to some embodiments, the terminal base of the 5′ end of the chimeric padlock probes is phosphorylated or is pre-adenylated before hybridization to the RNA sample. Hereby, the task of the ligase in ligation step may be varied, and in some situations facilitated and improved.

According to a second aspect there is provided a kit for use in in situ profiling of single nucleotide variations in an RNA sample, comprising at least one nucleotide position to be profiled, flanked by nucleotide stretches of known identity both at the 3′ and the 5′ side of the at least one nucleotide position to be profiled, comprising:
one or more chimeric padlock oligonucleotides having a 3′ end and a 5′ end, which 3′ end and 5′ ends are designed to hybridize to the RNA sample to the nucleotide stretch of known identity at the 3′ and 5′ sides of the at least one nucleotide position to be profiled, respectively, so that the 3′ terminal base, or any of the two bases next to the 3′ terminal base of the 3′ end, is designed to be positioned at the nucleotide position to be profiled in the RNA sample when the padlock probe is hybridized to the RNA sample, and wherein the 3′ end portion of said padlock oligonucleotides comprises a modified base at the 3′ end portion, wherein the modified base is chosen from C, A, G and U/T;
an RNA ligase having single nucleotide specificity, optionally including necessary reagents and buffers;
optionally one or more amplification primers and a polymerase for rolling circle amplification, and necessary reagents and buffers;
instructions for use.

Hereby, a kit for performing including the necessary reagents and instructions for performing the method described in this disclosure is provided.

According to some embodiments, the kit comprises: (v) one or more fluorescent dyes and/or one or a plurality of RNA base or modified base specific detection oligonucleotides.

According to some embodiments, the chimeric padlock oligonucleotides comprise an anchoring sequence, and the kit further comprises means for immobilizing the chimeric padlock oligonucleotides via the anchoring sequence.

According to some embodiments, the KOD RNA ligase is a KOD ligase chosen from KOD1Rnl from Thermococcus kodarensis.

According to some embodiments, the chimeric padlock oligonucleotides are designed to profile more than one single nucleotide variation positioned on the same or different RNA sample molecules.

According to some embodiments, the modified base of the 3′ end portion of the chimeric padlock probes is chosen from (i) an RNA base, (ii) a 2′-O-methoxy-ethyl base, (iii) a 2′-O-methyl RNA base, (iv) a 2′-fluoro base, (v) a DNA base, or (vi) an LNA base.

According to some embodiments, the modified base at the 3′ end portion is positioned within at least two bases from the 3′ terminal base.

According to some embodiments, the padlock probe has any of the following designs:
(i) the 3′ terminal end base is a modified base and is positioned at the nucleotide position to be profiled of the RNA sample;
(ii) the 3′ terminal end base is a modified base, and the base next to the 3′ terminal end base is positioned at the nucleotide position to be profiled of the RNA sample; or
(iii) the base next to the 3′ terminal end base is a modified base and is positioned at the nucleotide position to be profiled of the RNA sample.

According to some embodiments, the 5′ terminal base of the 5′ end is phosphorylated or pre-adenylated.

Hence, at least the following novel advantages and features are provided by the method described in this disclosure:
Previously, there is no reliable solution for in situ SNV detection on RNA.
The present method enables an about 5 times higher detection efficiency compared to methods requiring cDNA synthesis.
The present method is potentially compatible with FFPE samples, whereas cDNA-ISS (in situ sequencing) struggles with FFPE samples.
As a result of the present disclosure, applications as a solution to further immuno-oncology research and diagnostics can be provided.

Effects and features of the second aspect are to a large extent analogous to those described above in connection with the first aspect. Embodiments mentioned in relation to the first aspect are largely compatible with the second aspect.

The present disclosure will become apparent from the detailed description given below. The detailed description and specific examples disclose preferred embodiments of the disclosure by way of illustration only. Those skilled in the art understand from guidance in the detailed description that changes and modifications may be made within the scope of the disclosure.

Hence, it is to be understood that the herein disclosed disclosure is not limited to the particular component parts of the device described or steps of the methods described since such device and method may vary. It is also to be understood that the terminology used herein is for purpose of describing particular embodiments only, and is not intended to be limiting. It should be noted that, as used in the specification and the appended claim, the articles “a”, “an”, “the”, and “said” are intended to mean that there are one or more of the elements unless the context explicitly dictates otherwise. Thus, for example, reference to “a unit” or “the unit” may include several devices, and the like. Furthermore, the words “comprising”, “including”, “containing” and similar wordings does not exclude other elements or steps.

### Definitions

The term “chimeric” in the context of padlock probes refers to probes that include parts from different sources, such as a nucleic acid probe containing both DNA and RNA bases, e.g. DNA probe wherein the 3′ end is replaced with an RNA base or a 2-o-methyl RNA base.

The term “in-situ profiling” means measuring the abundance of variants (such as SNVs) in a biological sample.

The term “sample” is to be interpreted as any biological tissue sample from any species, and/or cultured cells on e.g. microscope slides or coverslips.

The term “ISS” is an abbreviation of “in-situ sequencing”.

The term “PLP” is an abbreviation of “padlock probe”.

The term “RCA” is an abbreviation of “rolling circle amplification”.

The terms “SNP” and “SNV” are abbreviations of “single nucleotide polymorphism” and “single nucleotide variation”, respectively. These terms both refer to variations/mutations of a single nucleotide position compared to a reference sequence (e.g. a wildtype), and in the context of this disclosure the terms “SNP” and “SNV” are used interchangeably.

The term “LNA” refers to a locked nucleic acid (LNA), also known as bridged nucleic acid (BNA), and often referred to as inaccessible RNA, which can be either a modified RNA or DNA nucleotide in which the ribose moiety is modified with an extra bridge connecting the 2′ oxygen and 4′ carbon. The bridge “locks” the ribose in the 3′-endo (North) conformation, which is often found in the A-form duplexes.

### BRIEF DESCRIPTIONS OF THE DRAWINGS

The above objects, as well as additional objects, features and advantages of the present disclosure will be more fully appreciated by reference to the following illustrative and non-limiting detailed description of example embodiments of the present disclosure, when taken in conjunction with the accompanying drawings.

FIG. 1 discloses an example of a padlock probe according to a prior art approach using cDNA as a template.

FIG. 2 illustrates cDNA-based ISS (in-situ sequencing) (Gyllborg et al., “Hybridization-based in situ sequencing (HybISS) for spatially resolved transcriptomics in human and mouse brain tissue”, Nucleic Acids Research, Volume 48, Issue 19, 4 Nov. 2020, Page e112, https://doi.org/10.1093/nar/gkaa792) for SNP profiling.

FIG. 3 shows a comparison of cDNA-based (1) and the RNA-based (2) approaches according to the present disclosure.

FIG. 4 shows a comparison of cDNA-based ISS (left) and the RNA-based ISS (right) according to the present disclosure.

FIG. 5 shows the principles of one embodiment of the present disclosure, including competing chimeric padlock probes and subsequent detection.

FIG. 6 (A) discloses the general design of a chimeric padlock probe for use in the present disclosure. In (B) its hybridization to an mRNA template is shown.

FIG. 7-10 shows single nucleotide variation (SNV) detection with KOD ligase.

FIG. 11-14 shows competing padlock probes on a known GPDH sequence in A549 cells.

FIG. 15 shows alternative designs of the padlock probe including 5′ modifications.

FIG. 16 shows alternative designs of the padlock probe including 3′ modifications.

### DETAILED DESCRIPTION

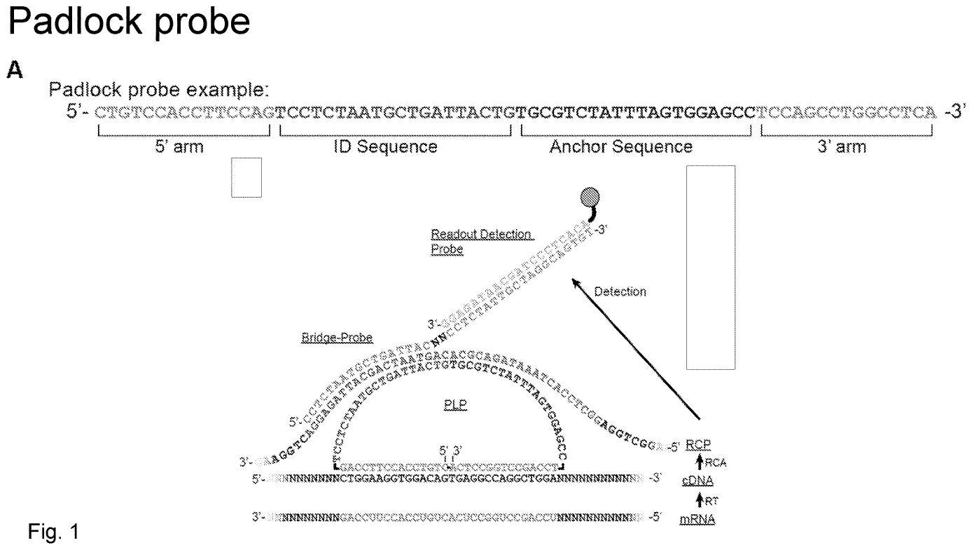

The present inventors have developed an approach, including methods and kits, using chimeric padlock probes in combination with a single-nucleotide specific ligase, which probes the single-nucleotide variation/polymorphism directly on the RNA molecule. Hereby, the issues of spatial resolution and increased detection sensitivity are addressed, as compared to the traditional cDNA-based approach. The overall principles of the present approach, compared to a prior art solution, will now be described with reference to FIGS. 1-6.

FIG. 1 discloses an example of a padlock probe in a prior art approach, wherein the padlock probe is designed to hybridize to a cDNA molecule, created by reverse transcription from a mRNA molecule of interest. The various typical parts of the padlock probe are shown, including a 5′ arm for hybridization to the template molecule at the 5′ side of a position to be identified, an ID sequence and an anchor sequence for use as templates for rolling circle amplification, and a 3′ arm for hybridization to the template molecule at the 3′ side of a position to be identified. The 3′ arm includes an RNA base at the end, for ligation purposes. A bridge-probe and a readout detection probe are designed to be hybridized to the amplified probe for detection purposes.

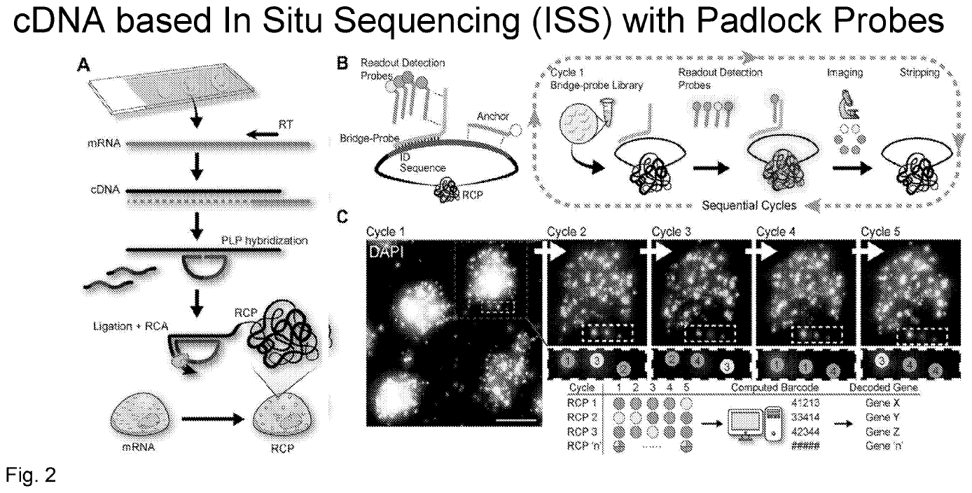

FIG. 2 illustrates cDNA-based ISS (in-situ sequencing) including the necessary steps from (A) mRNA extraction, reverse transcription, PLP hybridization, ligation and rolling circle amplification, to (B) and (C) detection in sequential cycles enabling multiple rolling circle products to be profiled.

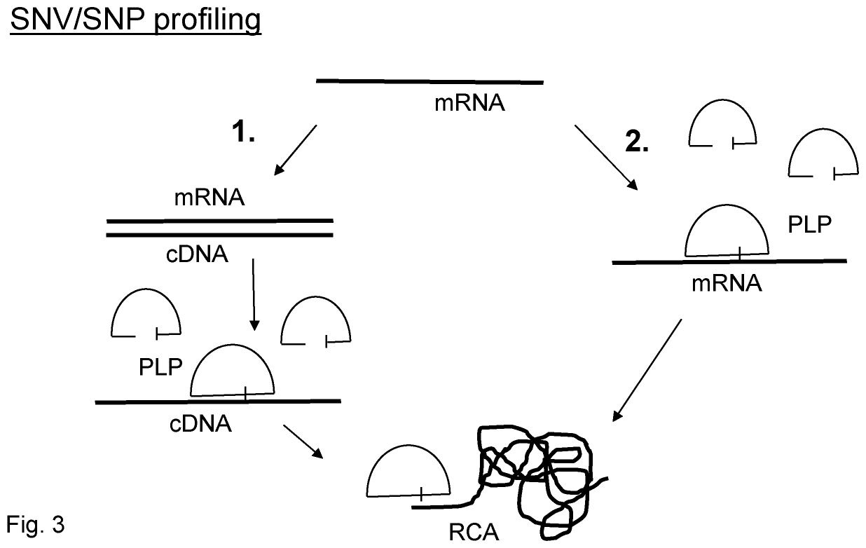

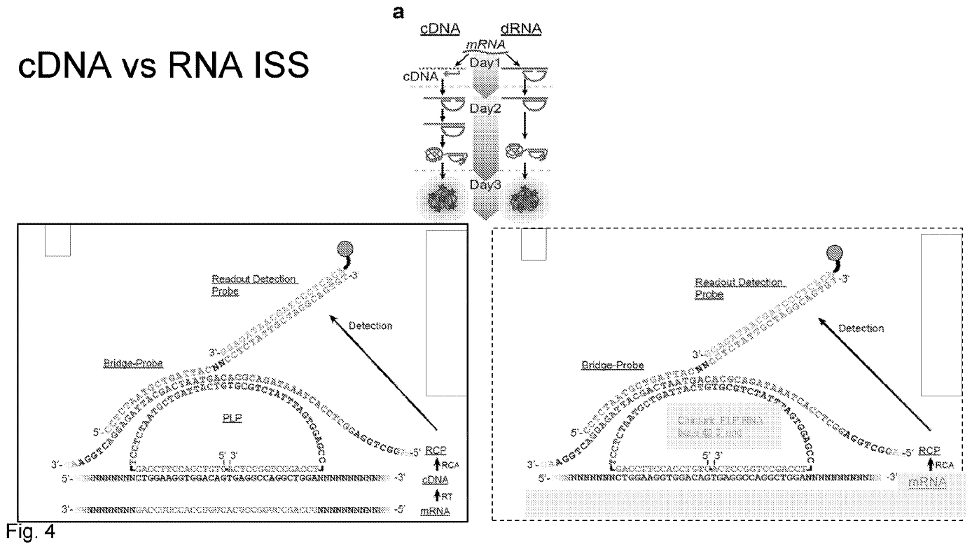

FIG. 3 illustrates cDNA-based ISS vs the newly developed direct mRNA targeting approach for SNP profiling of the present disclosure.
1) illustration of cDNA-based ISS where first cDNA is synthesized in situ from mRNA, before native mRNA is being digested, allowing the single stranded cDNA to be probed using competing DNA padlock probes (PLP). The correct PLP is then hybridized to the cDNA, before the ligation event of a 100% matched PLP, followed by amplification by rolling circle amplification.
2) illustration of the improved chemistry wherein competing chimeric PLPs are directly hybridized to the mRNA in situ, following a ligation of only the correct probe with KOD Ligase before RCA.

In both illustration 1 and 2, the rolling circle amplification products generated can then be fluorescently labeled with fluorescent oligonucleotides, which can then be visualized under the microscope for a fluorescent readout.

FIG. 4 shows comparisons of cDNA-based ISS (left) and the RNA-based ISS (right) according to the present disclosure. The RNA-based ISS omits one step (reverse transcription of mRNA to cDNA) as compared to cDNA-based ISS.

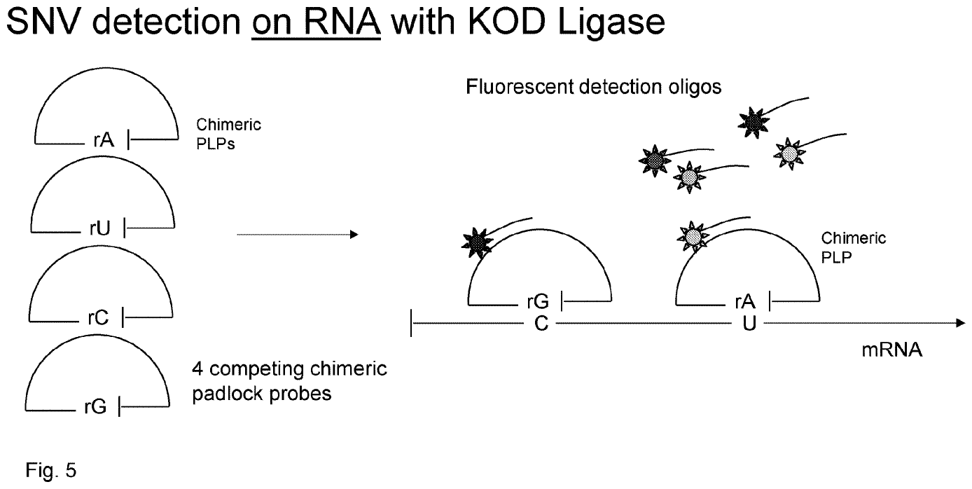

FIG. 5 shows the principles of the approach of the present disclosure, wherein competing chimeric padlock probes hybridize to the mRNA template, become ligated (not shown), amplified (not shown) and subsequently detected by means of fluorescence. In accordance with this embodiment, it is shown that a plurality of single-nucleotide variations in the RNA template molecule can be profiled, as long as the size of the padlock probes permit. Typically, a nucleotide distance of at least 30 nucleotides between two single-nucleotide variations on the same RNA molecule is necessary. As long as this criterium is met, there is no upper limit with regards to the number of single-nucleotide variations to profile in one single experiment.

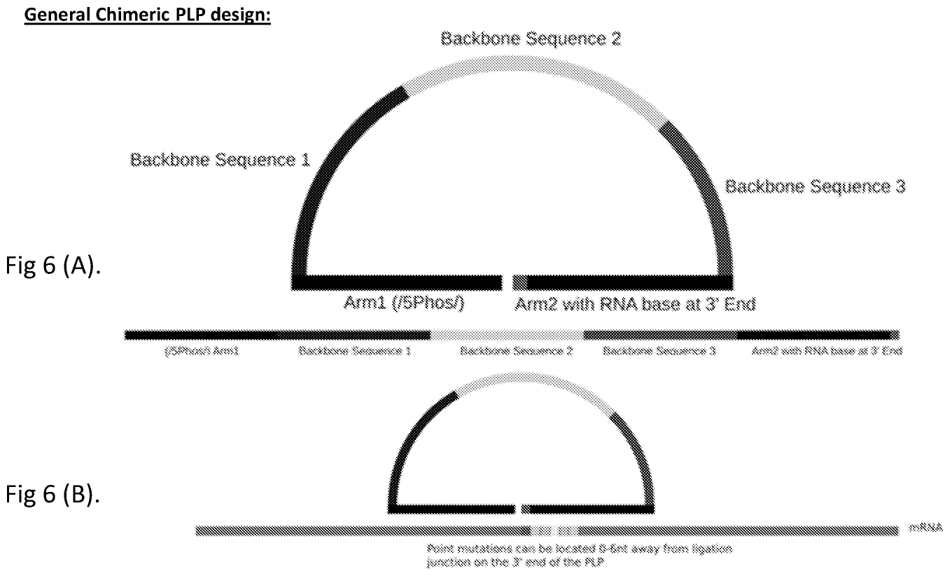

FIG. 6 (A) discloses the general design of a chimeric padlock probe for use in the present disclosure. In (B) its hybridization to an mRNA template is shown, also showing that single nucleotide variations in the mRNA, such as point mutations, can be located about 0-6 nucleotides away from the ligation junction on the 3′-end of the padlock probe.

Thus, the first aspect of this disclosure shows a method for in situ profiling of single nucleotide variations in an RNA sample, comprising the steps of:
(a) contacting the RNA sample with a plurality of chimeric padlock probes comprising an RNA base at the 3′ end, wherein the RNA base is chosen from C, A, G and U under conditions and with reagents allowing hybridization;
(b) adding an RNA ligase under conditions and with reagents allowing ligation of the padlock probes with a single nucleotide specificity, in order to generate a circularized padlock oligonucleotide for padlock probes comprising an RNA base that is complementary with the corresponding position of the RNA sample;
(c) amplifying the circularized padlock oligonucleotide under conditions and with reagents allowing rolling circle amplification, thereby generating an amplified circularized padlock oligonucleotide;
(d) detecting the amplified circularized padlock oligonucleotide, thereby obtaining a profile of single nucleotides in the RNA sample.

### The RNA Sample

In one embodiment, the RNA sample comprises at least one nucleotide position to be profiled, flanked by nucleotide stretches of known identity at the 3′ and the 5′ side of the at least one nucleotide position to be profiled.

In one embodiment, the RNA sample is an mRNA sample.

The RNA sample can be obtained from any tissue or cell line of choice, and e.g. be pretreated as exemplified in Example 1. The skilled person would be aware of alternative ways of extracting and/or pretreating the sample material.

For example, tumor sections are of interest to profile for mutations in the tumor micro environment for immune-oncology applications. However, a plurality of other applications can also be contemplated.

In order for the RNA sample to be used in the method of the present disclosure, at least parts of the RNA sample must include a known sequence of nucleotides, so that a padlock probe (as explained below) can be designed to hybridize to the RNA sample. At least, stretches of nucleotide of the RNA sample corresponding to the length of the parts of the padlock probes that are designed to hybridize to the RNA sample (i.e. the 3′ and 5′ arms) must be known. Also, the RNA sample comprises at least one nucleotide position to be profiled, i.e. a position wherein a single nucleotide variation (or polymorphism) (SNV/SNP) of interest is to be identified. Thus, the SNV must be flanked at both sides by known sequences, so that a padlock probe can be designed to hybridize both 3′ and 5′ to the SNV.

In one embodiment, the RNA sample comprises at least two single nucleotide variations (SNVs) on different positions in the RNA sample. In this case, different padlock probes must be designed for each SNV. The at least two SNVs can either (1) be positioned at such distance that at least two different padlock probes, designed for each SNV, simultaneously hybridize to the nucleotide area flanking each SNV. In this case, the nucleotide distance between the two SNVs would have to be at least the length of the 3′-arm of one of the padlock probes and the length of the 5′-arm of the other padlock probe, i.e. about 15+15 nucleotides. Otherwise, the at least two padlock probes cannot hybridize simultaneously to the same RNA sample molecule. Alternatively (2), the at least two SNVs can be positioned at a closer distance, meaning that only one of the at least two different padlock probes can hybridize for each individual RNA molecule, which would mean that the at least two different padlock probes would compete for hybridizing to the RNA sample, which typically would result in a lower detection signal. Thus, if the SNVs are positioned too closely together (at a distance of less than about 30 nucleotides), a lower detection efficiency in detecting the two SNVs simultaneously would typically be observed.

### Padlock Technology/Padlock Probes

In one embodiment, the plurality of chimeric padlock probes each have a first end and a second end, which first and second ends are designed to hybridize to the RNA sample to the nucleotide stretches of known identity at the 3′ and 5′ sides of the at least one nucleotide position to be profiled, respectively, so that the RNA base at the 3′ end is positioned at the nucleotide position to be profiled in the RNA sample.

In one embodiment, the padlock probes having an RNA base at the 3′end that is complementary with the nucleotide in the nucleotide position to be profiled of the RNA sample are ligated in step (c), and wherein padlock probes having an RNA base at the 3′end that is not complementary with the nucleotide in the nucleotide position to be profiled of the RNA sample are not ligated in step (c).

The general design of chimeric padlock probes is as shown in FIGS. 1 and/or 6. The technology of designing chimeric padlock probes in general is known in the art.

For this context, the choice and design of padlock probes need to take account for the one or more SNVs to be profiled. Some different situations may occur which can vary the choice of padlock probes to design and use:

In one situation, the RNA sample comprises one SNV to be profiled, wherein the identity of this SNV is unknown and can vary between all four bases (A, G, C, U). In this case, typically four padlock probes of the same overall design, but with different RNA bases at the 3′ end, will be designed and used. Upon use, the padlock probe having the RNA base that is complementary to the SNV of the RNA sample to be identified will completely hybridize to the RNA sample, including the 3′ end (i.e. U will hybridize with A, A with U, C with G and G with C), and subsequently be ligated, amplified and detected. The other padlock probes, comprising an RNA base at the 3′ end that is not complementary with the SNV of the RNA sample, will typically hybridize (at least partly), but will include a mismatch at the SNV position, and will therefore not be ligated, amplified and detected. Thus, detection will only occur for the padlock probe including an RNA base at the 3′end that base pairs with the SNV, and hence the identity of the SNV is determined.

In another situation, the RNA sample comprises one SNV to be profiled, wherein the identity of this SNV is partly unknown and is expected to vary between less than four bases, e.g. two bases. In this case, typically two padlock probes of the same overall design, but with different RNA bases at the 3′ end, will be designed and used. The padlock probes designed and used will include RNA bases at the 3′ end that are complementary to the expected identity of the SNV, i.e. if for example the SNV is expected to be either A or G, the padlock probes designed will include RNA base U and C at the 3′ end. In this case, there is no need to design padlock probes including RNA bases that are not expected to be complementary to the SNV, and costs can be saved. Upon use, the padlock probe having the RNA base that is complementary to the SNV of the RNA sample to be identified will completely hybridize to the RNA sample, including the 3′ end (i.e. U will hybridize with A, A with U, C with G and G with C), and subsequently be ligated, amplified and detected. The other padlock probe, comprising an RNA base at the 3′ end that is not complementary with the SNV of the RNA sample, will typically hybridize (at least partly), but will include a mismatch at the SNV position, and will therefore not be ligated, amplified and detected.

In yet another situation, the RNA sample may comprise more than one SNV to be profiled, and in this case more than one padlock probe can be designed and used. The overall principles of situation (1) and (2) will be applicable here as well. However, in order for different padlock probes to be able to hybridize simultaneously to different SNVs on the same RNA sample molecule, the padlock probes must be designed so that the nucleotide length of the hybridizing parts of the padlock probes (i.e. the 3′ and 5′ arms) allows simultaneous binding, i.e. so that the length of the 3′ arm of one padlock probe+the length of the 5′ arm of the other padlock probe is not shorter than the nucleotide distance between the at least two SNVs.

In yet another situation, there are multiple SNVs/mutations to profile across multiple transcript targets, thus a pool of wild type and mutant chimeric padlock probes can be designed and simultaneously used to probe for the multiple SNVs. The identity of these mutations profiled spatially can then all be extracted from a single experiment.

For typical conditions allowing hybridization of padlock probes to the RNA sample, details are provided below in the example section.

For the subsequent detection of the identity of the SNV to be profiled, detection oligonucleotides, possibly in combination with a bridging oligonucleotide (or bridge-probe) may be used. Typically, the padlock oligonucleotide is designed to comprise an ID sequence, which is specific for different padlock probes depending on the identity of the RNA base at the 3′ end, i.e. so that the specific ID sequence is unique for each possible alternative identity of the SNV position to be profiled. Hence, upon use, the bridging oligonucleotide being complementary to the ID sequence of the amplified circularized padlock oligonucleotide that is present will hybridize to the ID sequence. A detection oligonucleotide comprising a fluorescently signaling molecule, which provides a signal specific for the RNA base at the 3′ end of the ligated padlock probe (and hence of the identity of the SNV in the RNA sample), will then be possible to detect.

In some embodiments, an anchoring sequence is also included in the padlock oligonucleotide, for accessing amplification events, for a quick quality control.

An example of conditions typical for padlock hybridization according to the present method is shown in the example section, wherein PLP hybridization is performed at 37° C. (the temperature can be varied between about 37-55° C.) overnight. Hybridization may or may not be followed by washing steps to eliminate the excess of unhybridized probes. Also, additional blocking reagents such as salmon sperm DNA could be supplemented as an optional step. Salmon Sperm DNA Solution was developed for use in hybridization protocols as a blocking agent to reduce the non-specific binding of a hybridization probe (https://assets.fishersci.com/TFS-Assets/LSG/manuals/15632011.pdf). To the same purpose, an alternative blocking reagent to salmon sperm DNA is yeast tRNA (https://assets.thermofisher.com/TFS-Assets % 2FLSG %2Fmanuals %2Fsp 7119.pdf).

### Modifications and Alternative Designs of the Padlock Probe

In some designs of the padlock probes, the 3′ and/or the 5′ terminal ends of the padlock probe may be modified. By modifying the terminal ends of the padlock probe, the single nucleotide specificity can often be improved. Also, by using base modification that the ligase accepts for SNP profiling purposes, the cost of probes can potentially be reduced. Moreover, ligation efficiency may be increased.

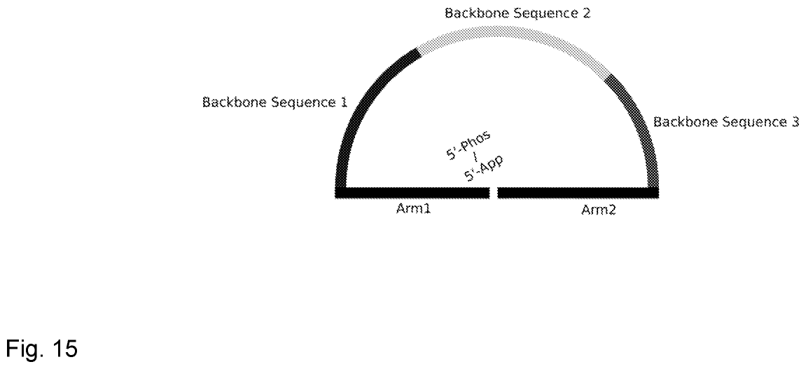

With regard to 5′ modification (FIG. 15), the 5′ end of the probes used can be pre-adenylated before hybridization to the RNA sample instead of a 5′ phosphate group. Typically, PLPs are provided 5′ phosphorylated. However, ligation occurs in two steps: first the ligase adenylates the 5′ end before catalysing the formation of a phosphodiester bond. The inventors have improved this process, by first adenylating the probes, by using a 5′ adenylation kit, and thereafter hybridizing these adenylated probes. This way, the ligase only has to catalyse one step instead of two, potentially increasing the efficiency of the ligation, and therefore of the entire method.

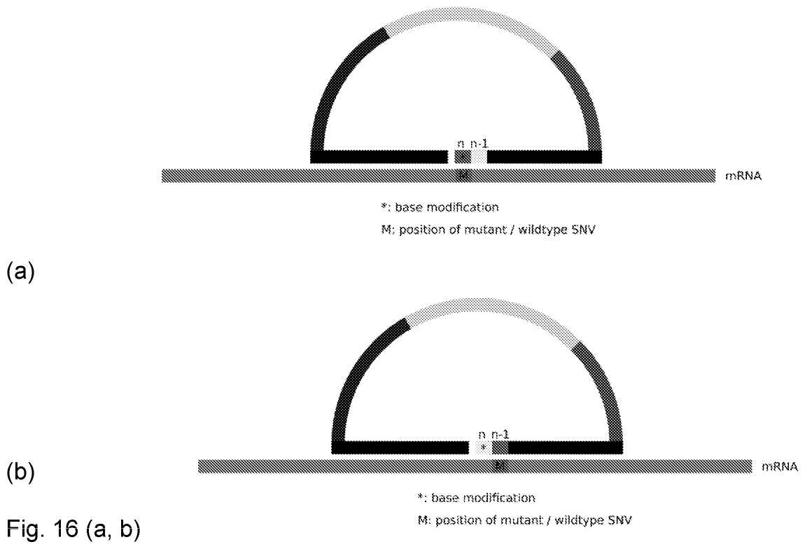

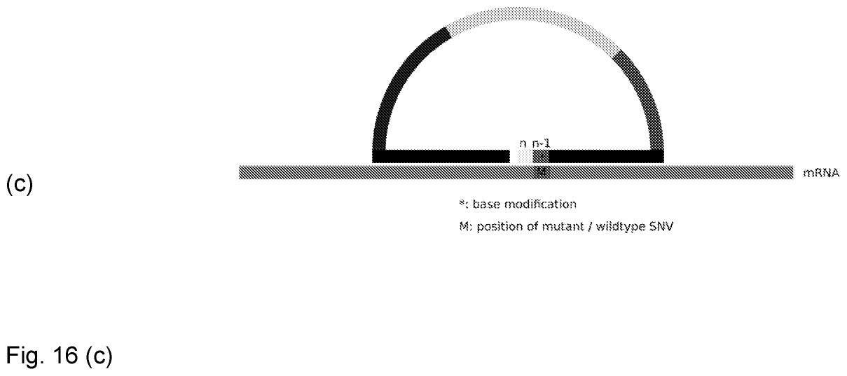

With regard to 3′ modification (FIG. 16 (a-c)), the following alternatives are provided: (a) With the mutant/wild type (nucleotide to be profiled) base at the 3′ terminal of the probe (position n), the 3′ terminal base (position n) modification can be:
RNA base (A, G, C or U)
2′-O-methoxy-ethyl bases (2′-MOE)
2′-O-Methyl RNA bases
2′ Fluoro bases
Unmodified DNA bases
LNA base
(b) With the mutant/wild type (nucleotide to be profiled) base located at the position next to the 3′ terminal of the probe (n-1), the terminal base at 3′ (position n) carries a base modification of:
RNA base (A, G, C or U)
2′-O-methoxy-ethyl bases (2′-MOE)
2′-O-Methyl RNA bases
2′ Fluoro bases
Unmodified DNA base
LNA bases
(c) With the mutant/wild type (nucleotide to be profiled) base located at the position next to the 3′ terminal of the probe (n-1), the base at position (n-1) carries a base modification of:
RNA base (A, G, C or U)
2′-O-methoxy-ethyl bases (2′-MOE)
21-O-Methyl RNA bases
2′ Fluoro bases
Unmodified DNA base
LNA bases
(d) With the mutant/wild type (nucleotide to be profiled) base located at the position two bases from the 3′ terminal of the probe (n-2), the mutant/wild type base at position (n-2) carries a base modification of:
RNA base (A, G, C or U)
2′-O-methoxy-ethyl bases (2′-MOE)
2′-O-Methyl RNA bases
2′ Fluoro bases
Unmodified DNA base
LNA bases

In embodiments where the base is located at position (n-1) or (n-2), the base in position n or in position n and (n-1), respectively, is typically a standard DNA base. Typically, positions of the probe not having a modified base, typically is a standard/normal DNA base.

The probe modifications listed above have been shown to be compatible with the function of the ligases used and suggested in the present disclosure for the purposes of SNP detection. Some of these probe modifications represent previously undisclosed probe alternatives for applications of this type.

LNA bases has the potential advantage that LNA is able to offer improved specificity in base pairing and that will allow for the correct probe to hybridize better than a mismatched probe, facilitating for the ligase to discriminate perfect vs imperfect hybridization before ligation.

### Ligation

One key feature of the present invention is to use an RNA ligase having single-nucleotide specificity. Hereby, the specificity of the RNA ligase makes it possible to discriminate between padlock probes having a mismatch at the SNV (i.e. for which the RNA base at the 3′ end is not complementary to the SNV nucleotide identity) and padlock probes base-pairing to the SNV position (i.e. for which the RNA base at the 3′ end is complementary to the SNV nucleotide identity). For padlock probes base-pairing to the SNV position the RNA ligase will seal the 3′ and 5′ ends of the padlock oligonucleotide thereby producing a circularized padlock oligonucleotide, which subsequently undergoes amplification and detection to determine the identity of the SNV position.

The single nucleotide specificity can e.g. be validated by a validation model based on a sequencing database where known isoforms/SNPs/SNVs (single nucleotide polymorphisms/variations) are targeted. Also, the specificity of the approach disclosed in this specification can be validated by running a control experiment using cDNA-ISS where previously it has been shown to be able to profile mutations on cDNA generated from mRNA in situ. The results obtained correlate very well with cDNA-ISS data.

In some embodiments, the single-nucleotide specificity can be quantified in situations requiring that only a 100% correct matching hybridization of the padlock probe is ligated. Various modifications at the 3′ end, such as those listed in this disclosure, can facilitate for the ligase to discriminate between a perfectly (100%) hybridized construct vs a mismatched (less than 100%) hybridization before ligation can occur.

In one embodiment the RNA ligase is a KOD RNA ligase, chosen from KOD1Rnl from Thermococcus kodarensis. This RNA ligase has shown to be able to ligate padlock probes with a sufficiently high accuracy.

One important criterium for selecting a ligase that have the potential to work in the method of the present disclosure, is that the ligase has the ability to accept RNA as a splint to catalyze a ligation reaction.

In some embodiments, the ligase is chosen from PBCV-1 DNA Ligase/Chlorella virus DNA Ligase, engineered ligases from the PBCV-1 family, and engineered ligases from the family of archaeon, Thermococcous kodarensis.

For typical conditions allowing ligation with KOD RNA ligase, details are provided below in the example section. The conditions may of course vary depending on choice of ligase. Typically, the choice of the ligase will determine the other necessary conditions. Followed by the choice of ligase, monovalent and divalent salt concentration and pH are the most important parameters. For KOD ligase, the optimal pH is reported to be about 7.5, and therefore a pH in the interval of about 7-8 is typically suitable for the ligation step.

### Rolling Circle Amplification

Circularized padlock probes, i.e. padlock probes that have undergone ligation, and therefore includes an RNA base at the position of the ligated 3′-end that is complementary to the SNV position to be identified, will undergo amplification, typically using rolling circle amplification.

For typical conditions allowing rolling circle amplification, details are provided below in the example section.

In some embodiments, the primers, enzymes and other reagents for the steps of hybridization, ligation and amplification (typically RCA) may be added in separate steps, or at least partly combined, thereby increasing efficiency of the method. For example, RCA primers may be added to the ligation mix to anneal to the hybridized chimeric PLPs. Also, competing PLPs can also be added to the ligation mix.

### Detection

The detection of amplified circularized padlock probes in order to profile the SNV or SNVs to be identified can be performed in several different ways, for example by means of luminescence, such as fluorescence, and/or by means of sequencing, such as Sequencing by Hybridization SBH, Sequencing by Ligation SBL, SOLID sequencing or Sequencing by Synthesis SBS.

In one embodiment, detection oligonucleotides including means for luminescent signaling are added to the amplified circularized padlock oligonucleotides, wherein the detection oligonucleotides are designed to bind directly to the amplified circularized padlock oligonucleotides in an RNA base specific manner, or to a bridging oligonucleotide that is designed to bind to the amplified circularized padlock oligonucleotides in an RNA base specific manner, so that the resulting luminescent signal allows profiling of the single nucleotide variation in the RNA sample.

In one embodiment, the bridging oligonucleotide is specific for an ID sequence included in the padlock probe. The ID sequence is specific for different padlock probes, depending on the identity of the RNA-base at the 3-end (i.e. whether the RNA base is U, A, C or G), and hence only a bridging oligonucleotide corresponding to a certain RNA base at the 3-end of the padlock oligonucleotide to which it hybridizes will hybridize. This way, only amplified circularized oligonucleotides will be possible to detect. Typically, this means that bridging oligonucleotides corresponding to all possible SNV variations should be added in order to obtain a detectable signal. For example, in the case of four (4) different possible SNV variations (A, C, U, G) this means that four (4) different bridging oligonucleotides should be added.

Also, detection oligonucleotides that are specific for each alternative bridging oligonucleotide are added, wherein each detection oligonucleotide comprises luminescence (such as fluorescence) signaling molecule. Upon binding of the specific detection oligonucleotide to the bridging oligonucleotide to which it is specific, which in turn specifically hybridizes with an amplified circularized padlock oligonucleotide depending on identity of RNA base at the 3′ end (and hence of the identity of the SNV to be profiled), a detection signal unique for the identity of the SNV to be profiled can be detected. Typically, this means that detection oligonucleotides corresponding to all available bridging oligonucleotides should be added in order to obtain a detectable signal. For example, in the case of four (4) available different bridging oligonucleotides, four (4) different detection oligonucleotides providing different detection signals, should be added.

For a situation where the RNA sample comprises more than one SNV to be profiled, the detection can be performed by adding an additional, unique, set of detection oligonucleotides and/or bridging oligonucleotides, thereby providing additional detection signals that make it possible to discriminate between different SNVs, in addition to profiling the identity of each SNV. Alternatively, detection can be performed in cycles, wherein for each cycle the identity of one SNV is profiled.

Details on various options and alternatives in this regard are available from the following reference: https://doi.org/10.1093/nar/gkaa792.

Hence, the luminescent signaling may include a fluorescent signal, which is detectable e.g. via a microscope or other imaging technology, thereby allowing the fluorescent signal to be detected.

In one embodiment, the profile of the amplified circularized padlock oligonucleotides is distinguished by fluorescent readout by introducing different barcodes in the backbone sequence of the padlock probes for the competing PLP probes. The backbone sequence design of competing PLPs (where the RNA base at the 3′ end can be either A, U, C or G) can have a unique backbone sequence that can be distinguished with fluorescent detection probes after RCA.

### Kit

In a second aspect of this disclosure, a kit for use in the first aspect for in-situ profiling of single nucleotide variations in an RNA sample is provided, comprising at least one nucleotide position to be profiled, flanked by nucleotide stretches of known identity both at the 3′ and the 5′ side of the at least one nucleotide position to be profiled, comprising:
one or more chimeric padlock oligonucleotides having a 3′ end and a 5′ end, which 3′ end and 5′ ends are designed to hybridize to the RNA sample to the nucleotide stretch of known identity at the 3′ and 5′ sides of the at least one nucleotide position to be profiled, respectively, so that the 3′ terminal base or the base next to the 3′ terminal base of the 3′ end is designed to be positioned at the nucleotide position to be profiled in the RNA sample when the padlock probe is hybridized to the RNA sample, and wherein the 3′ end portion of said padlock oligonucleotides comprises a modified base at the 3′ end portion, wherein the modified base is chosen from C, A, G and U/T;
an RNA ligase having single nucleotide specificity, optionally including necessary reagents and buffers;
optionally one or more amplification primers and a polymerase for rolling circle amplification, and necessary reagents and buffers;
instructions for use.

In one embodiment the kit comprises: (v) one or more fluorescent dyes and/or one or a plurality of RNA base specific detection oligonucleotides.

In one embodiment, the chimeric padlock oligonucleotides comprise an anchoring sequence, and the kit further comprises means for immobilizing the chimeric padlock oligonucleotides via the anchoring sequence.

In one embodiment the RNA ligase is chosen from the group comprising (i) KOD ligase chosen from KOD1Rnl from Thermococcus kodarensis, (ii) PBCV-1 DNA Ligase/Chlorella virus DNA Ligase, (iii) engineered ligases from the PBCV-1 family, and (iv) engineered ligases from the family of archaeon, Thermococcous kodarensis.

In one embodiment, the chimeric padlock oligonucleotides are designed to profile more than one single nucleotide variation positioned on the same or different RNA sample molecules.

In one embodiment, the modified base of the 3′ end portion of the chimeric padlock probes is chosen from (i) an RNA base, chosen from A, G, C and U, (ii) a 2′-O-methoxy-ethyl base, (iii) a 2′-O-methyl RNA base, (iv) a 2′-fluoro base, (v) a DNA base or (vi) an LNA base.

In one embodiment, the modified base at the 3′ end portion is positioned within at least two bases from the 3′ terminal base.

In one embodiment, the padlock probe has any of the following designs:
(i) the 3′ terminal end base is a modified base and is positioned at the nucleotide position to be profiled of the RNA sample;
(j) the 3′ terminal end base is a modified base, and the base next to the 3′ terminal end base is positioned at the nucleotide position to be profiled of the RNA sample; or
(k) the base next to the 3′ terminal end base is a modified base and is positioned at the nucleotide position to be profiled of the RNA sample.

In one embodiment, the 5′ terminal base of the 5′ end is phosphorylated or pre-adenylated.

Thus, a kit for use in the method of the present disclosure typically comprises necessary reagents for the overall method in general, and, often, specific probes and material for the specific profiling application.

The invention will now be described by the following examples, with the purpose of illustrating specific embodiments of the invention, and are not to be construed as limiting the invention in any way.

### EXAMPLES

The present disclosure will now be described with reference to the accompanying examples, in which preferred example embodiments of the disclosure are shown. The disclosure may, however, be embodied in other forms and should not be construed as limited to the herein disclosed embodiments. The disclosed embodiments are provided to fully convey the scope of the disclosure to the skilled person.

### Example 1: Protocol for SNV Detection with KOD Ligase & Chimeric Probes

The skilled person would understand that some reagents and conditions could be modified. The provided examples are thus for purposes of exemplifying the present invention.

### Sample Pretreatment

Fresh frozen biological samples (can be cell line/any tissue that has been sectioned onto a microscope slide/coverslip) is first fixed with 3.7% formaldehyde.

If the sample is FFPE samples, dewaxing/de-crosslinking is first performed with xylene and heat treatment (i.e. incubation at 45 degrees for 15 minutes).

The biological sample is then permeabilized with 0.1M HCl, with the addition of pepsin or proteinase K, or any other reagents typically used for permeabilization for FISH experiments.

### Chimeric Padlock Probe Design (See FIG. 5A for General Chimeric PLP Design).

First, a chimeric PLP design encompasses 2 arms (Arm1 & Arm2) that are complementary to the mRNA sequence of interest. The total combined length of Arm1 & Arm2 should be 30-50 nt in length (15-25 nt per arm for a symmetric design). It is also possible for one to design asymmetrical PLPs with a longer 5′ (arm1) and shorter 3′ (arm2).

The presence of an RNA base (rA, rU, rC or rG) on the terminal base of the 3′ Arm2 is a fundamental feature for some RNA ligases to act correctly.

Two (2) or more unique backbone sequences can be introduced by the user which includes a RCA priming site to allow for a RCA primer to anneal and potentially any other unique sequences to facilitate probe identification downstream after amplification.

Chimeric PLP Design to Profile Mutations with KOD Ligase (FIG. 5B):

To profile point mutations/SNPs/SNVs in situ on a mRNA of interest, the PLP can be designed in such a way that the point mutation sits anywhere from 0-6 nt length away from the ligation nick, on the 3′ end of the PLP.

### Hybridization

Chimeric padlock probes (PLPs) are then added to a hybridization buffer for PLP hybridization.

TABLE 1
Hybridization mixture.

Hybridization Mixture
2X SSC
20% Formamide or Ethylene Carbonate
10 nM of each PLP

PLP hybridization is performed at 37° C. (the temperature can be varied between about 37-55° C.) overnight. Hybridization may or may not be followed by washing steps to eliminate the excess of unhybridized probes. Also, additional blocking reagents such as salmon sperm DNA or yeast tRNA could be supplemented as an optional step.

### Ligation

TABLE 2
Example of KOD Ligase Ligation Buffer
composition and condition.

1X KOD Ligation Buffer
2 mM DTT
50 mM NaCl
50 mM Tris-HCl
10 mM MgCl₂
0.8 mM ATP
0.05 μM RCA primer
1 U/μl RiboProtect (RNase inhibitor)
0.1 μg/μl KOD Ligase

RCA primer (may also be added in the RCA mix below) is also spiked into the ligation mix to anneal to the hybridized chimeric PLPs. Competing PLPs can also be spiked into the ligation mix. Ligation is performed at 55° C. for 2 h.

The pH for the ligation step should be in the interval from 7-8.

### Rolling Circle Amplification (RCA)

TABLE 3
RCA reaction buffer composition.

RCA Buffer composition
50 mM Tris-HCl
10 mM MgCl₂
10 mM (NH4)₂SO₄
5% Glycerol
0.25 mM dNTPs
0.2 μg/ml BSA
0.05 μM RCA primer (of it is not
already added in the ligation mix)
1 U/μl Phi29 Polymerase

RCA primers can also be added in the RCA mix, if one did not previously add it into the ligation mix.

### Example 2-Competing Padlock Probes for Known SNV Detection on GAPDH

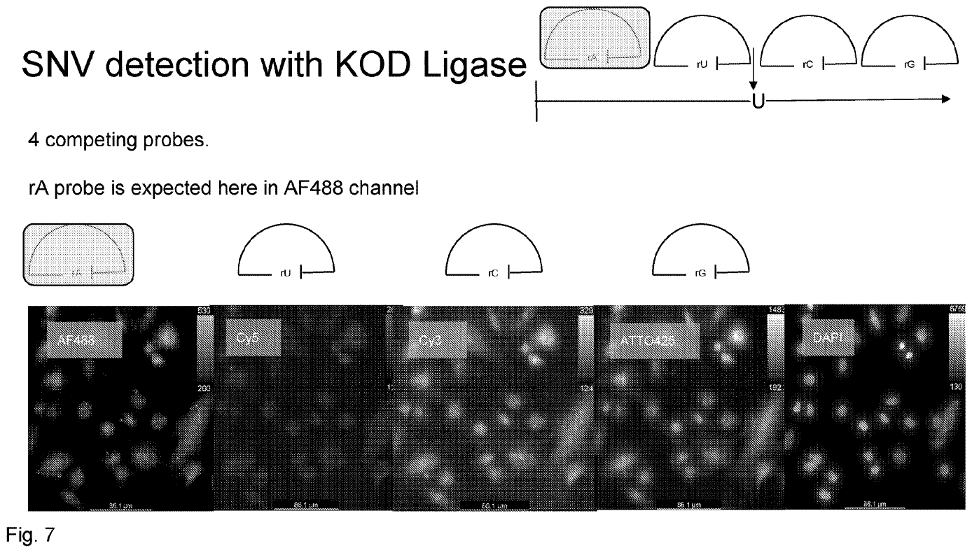

FIGS. 7-10 show detection of single-nucleotide variations with KOD ligase.

In FIG. 7, four competing probes are included, including “A”, “U”, “C” and “G”, respectively, at the 3′ end. The SNV to be profiled is “U”, meaning that the only padlock probe being ligated and giving rise to a detectable signal should be the one including an “A” at the 3′ end. A detectable signal is obtained in the detection channel corresponding to the “A” probe, i.e. AF488, but not in any other channel.

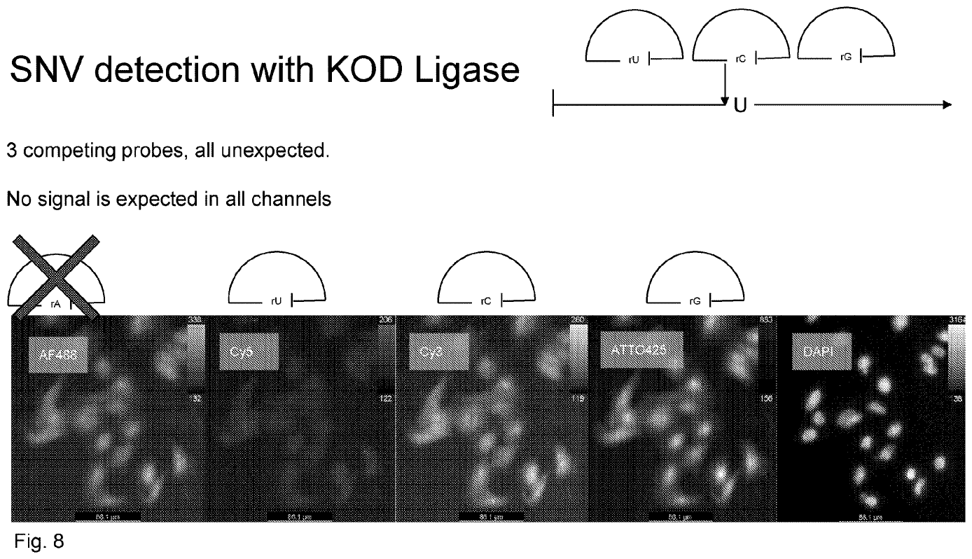

In FIG. 8, a negative control is performed. Only padlock probes not including an “A” at the 3′ end are included. The SNV to be profiled is “U”, meaning that no padlock probe is expected to be ligated and give rise to a detectable signal. As can be seen, no detectable signal is obtained in the AF488 channel, nor in any other channel.

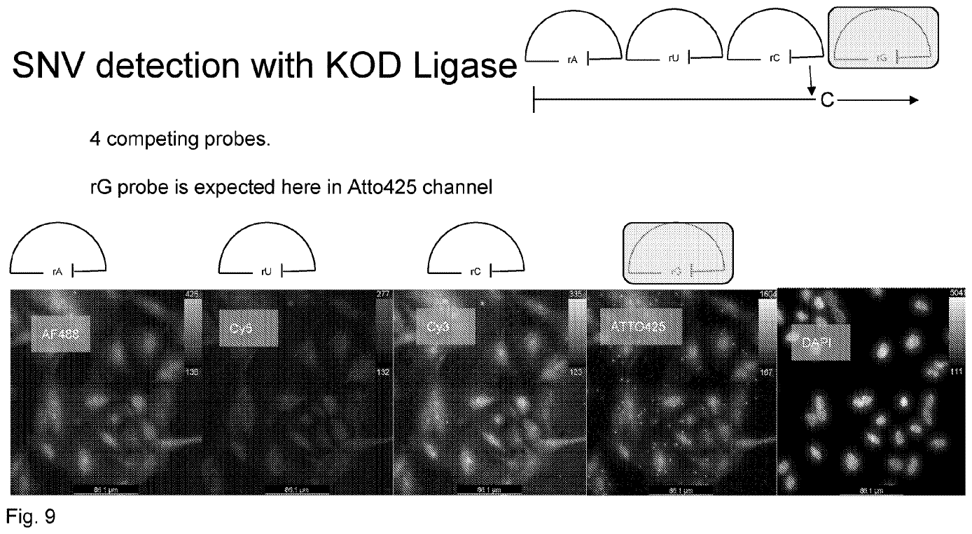

In FIG. 9, four competing probes are included, including “A”, “U”, “C” and “G”, respectively, at the 3′ end. The SNV to be profiled is “C”, meaning that the only padlock probe being ligated and giving rise to a detectable signal should be the one including a “G” at the 3′ end. A detectable signal is obtained in the detection channel corresponding to the “G” probe, i.e. Atto425, but not in any other channel.

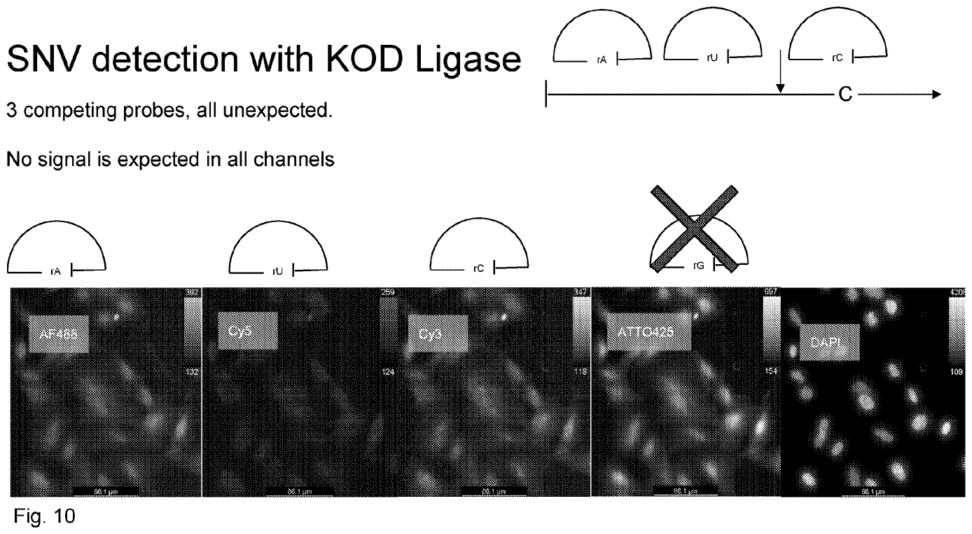

In FIG. 10, a negative control is performed. Only padlock probes not including a “G” at the 3′ end are included. The SNV to be profiled is “C”, meaning that no padlock probe is expected to be ligated. As can be seen, no detectable signal is obtained in the Atto425 channel, nor in any other channel.

### Example 3-Competing Padlock Probes on Known GAPDH Sequence in A549 Cells

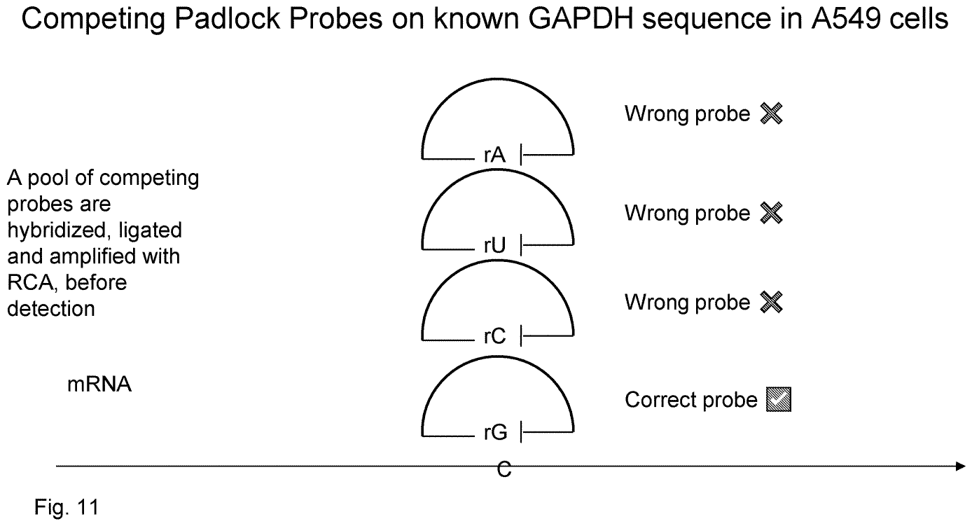

FIGS. 11-14 show the behavior of competing padlock probes on a known GAPDH sequence in A549 cells.

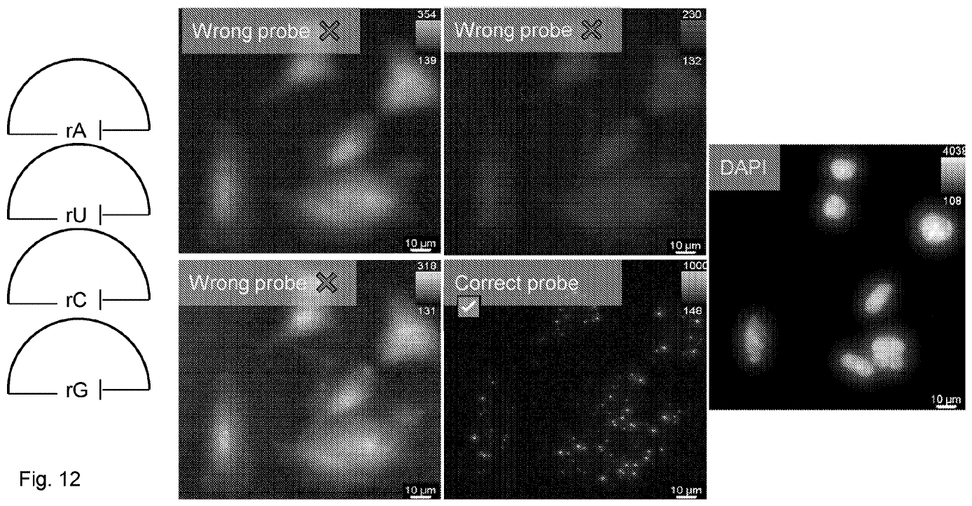

In FIG. 11, four competing probes are included, including “A”, “U”, “C” and “G”, respectively, at the 3′ end. The SNV to be profiled is “C”, meaning that the only padlock probe being ligated and giving rise to a detectable signal should be the one including a “G” at the 3′ end. FIG. 12 discloses detection results, wherein only the channel for the “G” padlock probe provides detectable signal.

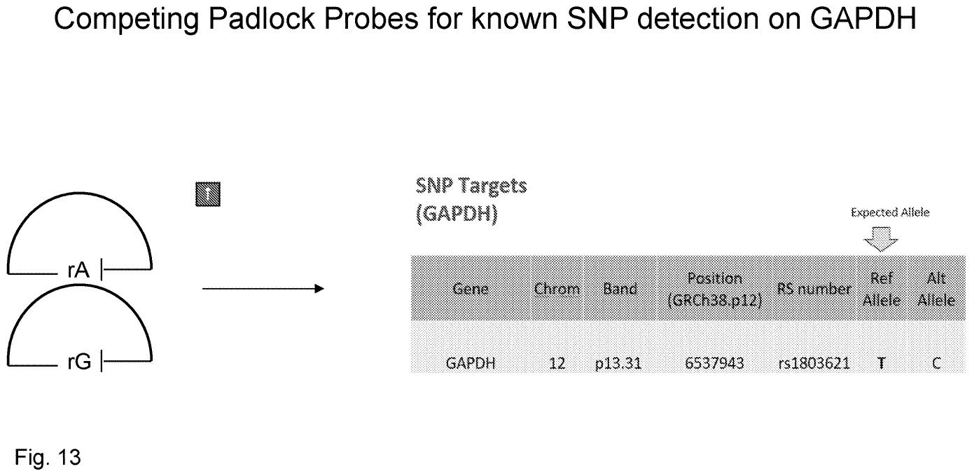

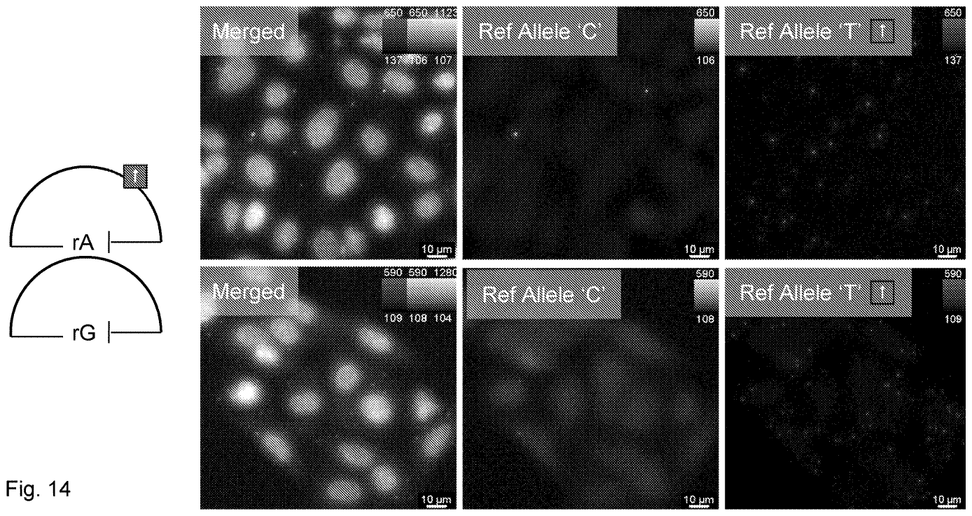

In FIG. 13, two competing padlock probes are included, “A” and “G”. The RNA sample (GAPDH) comprises two alleles for the SNP to be detected, wherein one allele is “T” and the other one is “C” in the position to be profiled. FIG. 14 discloses detection results showing that the “T” allele is more frequent, and the “C” allele is occurring, but at a lower rate.

The person skilled in the art realizes that the present disclosure is not limited to the preferred embodiments described above. The person skilled in the art further realizes that modifications and variations are possible within the scope of the appended claims. Additionally, variations to the disclosed embodiments can be understood and effected by the skilled person in practicing the claimed disclosure, from a study of the drawings, the disclosure, and the appended claims.

1. A method for in-situ profiling of single nucleotide variations in an RNA sample, comprising the steps of:
(a) contacting the RNA sample with a plurality of chimeric padlock probes, comprising a modified base at the 3′ end portion, wherein the modified base is chosen from C, A, G and U/T under conditions and with reagents allowing hybridization;
(b) adding an RNA ligase under conditions and with reagents allowing ligation of the padlock probes with a single nucleotide specificity, in order to generate a circularized padlock oligonucleotide for padlock probes comprising a modified base that is complementary with the corresponding position of the RNA sample;
(c) amplifying the circularized padlock oligonucleotide under conditions and with reagents allowing rolling circle amplification, thereby generating an amplified circularized padlock oligonucleotide;
(d) detecting the amplified circularized padlock oligonucleotide, thereby obtaining a profile of single nucleotides in the RNA sample.

2. The method according to claim 1, wherein the RNA sample comprises at least one nucleotide position to be profiled, flanked by nucleotide stretches of known identity at the 3′ and the 5′ side of the at least one nucleotide position to be profiled.

3. The method according to claim 1 or 2, wherein the RNA sample is chosen from an mRNA, an rRNA, a micro-RNA or a non-coding RNA sample.

4. The method according to claim 2 or 3, wherein the plurality of chimeric padlock probes each have a first end and a second end, which first and second ends are designed to hybridize to the RNA sample to the nucleotide stretches of known identity at the 3′ and 5′ sides of the at least one nucleotide position to be profiled, respectively, so that the terminal base at the 3′ end of the padlock probe, or any of the two bases next to the terminal base at the 3′ end, is positioned at the nucleotide position to be profiled in the RNA sample.

5. The method according to any one of claims 2-4, wherein padlock probes having bases at the 3′end portion that are complementary with the nucleotide in the nucleotide position to be profiled of the RNA sample are ligated in step (c), and wherein padlock probes having at least one base at the 3′end portion that is not complementary with the nucleotide in the nucleotide position to be profiled of the RNA sample are not ligated in step (c).

6. The method according to any of the preceding claims, wherein the RNA ligase is chosen from the group comprising (i) KOD ligase chosen from KOD1Rnl from Thermococcus kodarensis, (ii) PBCV-1 DNA Ligase/Chlorella virus DNA Ligase, (iii) engineered ligases from the PBCV-1 family, and (iv) engineered ligases from the family of archaeon, Thermococcous kodarensis.

7. The method according to any of the preceding claims, wherein the detection in step (e) is by means of luminescence, such as fluorescence, and/or by means of sequencing, such as Sequencing by Hybridization (SBH), Sequencing by Ligation (SBL), SOLID sequencing or Sequencing by Synthesis (SBS).

8. The method according to claim 7, wherein detection oligonucleotides are added to the amplified circularized padlock oligonucleotides, said detection oligonucleotides are designed to bind directly to the amplified circularized padlock oligonucleotides in an RNA base specific manner, or to a bridging oligonucleotide that is designed to bind to the amplified circularized padlock oligonucleotides in an RNA base specific manner, so that the resulting luminescent signal allows profiling of the single nucleotide variation in the RNA sample.

9. The method according to any of the preceding claims, wherein the profile of the amplified circularized padlock oligonucleotides is distinguished by fluorescent readout by introducing different barcodes in the backbone sequence of the padlock probes for the competing PLP probes.

10. The method according to any one of the preceding claims, wherein more than one single nucleotide variation positioned on the same RNA sample molecule are profiled.

11. The method according to any one of the preceding claims, wherein more than one single nucleotide variation positioned on different RNA sample molecules are profiled.

12. The method according to any one of the preceding claims, wherein the modified base at the 3′ end portion of the chimeric padlock probes is chosen from (i) an RNA base, (ii) a 2′-O-methoxy-ethyl base, (iii) a 2′-O-methyl RNA base, (iv) a 2′-fluoro base, (v) a DNA base or (vi) an LNA base.

13. The method according to any one of the preceding claims, wherein the modified base at the 3′ end portion is positioned within at least two bases from the 3′ terminal base.

14. The method according to claim 13, wherein the remaining bases of the 3′ end portion are non-modified DNA bases.

15. The method according to any one of the preceding claims, wherein the padlock probe has any of the following designs:
(i) the 3′ terminal end base is a modified base and is positioned at the nucleotide position to be profiled of the RNA sample;
(ii) the 3′ terminal end base is a modified base, and the base next to the 3′ terminal end base is positioned at the nucleotide position to be profiled of the RNA sample; or
(iii) the base next to the 3′ terminal end base is a modified base and is positioned at the nucleotide position to be profiled of the RNA sample.

16. The method according to any one of the preceding claims, wherein the terminal base of the 5′ end of the chimeric padlock probes is phosphorylated or is pre-adenylated before hybridization to the RNA sample.

17. A kit for use in in-situ profiling of single nucleotide variations in an RNA sample according to any one of claims 1 to 16, said RNA sample comprising at least one nucleotide position to be profiled, flanked by nucleotide stretches of known identity both at the 3′ and the 5′ side of the at least one nucleotide position to be profiled, comprising:
a. one or more chimeric padlock oligonucleotides having a 3′ end and a 5′ end, which 3′ end and 5′ ends are designed to hybridize to the RNA sample to the nucleotide stretch of known identity at the 3′ and 5′ sides of the at least one nucleotide position to be profiled, respectively, so that the 3′ terminal base or any of the two bases next to the 3′ terminal base of the 3′ end is designed to be positioned at the nucleotide position to be profiled in the RNA sample when the padlock probe is hybridized to the RNA sample, and wherein the 3′ end portion of said padlock oligonucleotides comprises a modified base at the 3′ end portion, wherein the modified base is chosen from C, A, G and U/T;
b. an RNA ligase having single nucleotide specificity, optionally including necessary reagents and buffers;
c. optionally one or more amplification primers and a polymerase for rolling circle amplification, and necessary reagents and buffers;
d. instructions for use.

18. The kit according to claim 17, further comprising: (v) one or more fluorescent dyes and/or one or a plurality of RNA base specific detection and/or bridging oligonucleotides.

19. The kit according to any one of claim 17 or 18, wherein the chimeric padlock oligonucleotides comprise an anchoring sequence, and the kit further comprises means for immobilizing the chimeric padlock oligonucleotides via the anchoring sequence.

20. The kit according to any one of claims 17 to 19, wherein the RNA ligase is chosen from the group comprising (i) KOD ligase chosen from KOD1Rnl from Thermococcus kodarensis, (ii) PBCV-1 DNA Ligase/Chlorella virus DNA Ligase, (iii) engineered ligases from the PBCV-1 family, and (iv) engineered ligases from the family of archaeon, Thermococcous kodarensis.

21. The kit according to any one of claims 17 to 20, wherein the chimeric padlock oligonucleotides are designed to profile more than one single nucleotide variation positioned on the same or different RNA sample molecules.

22. The kit according to any one of claims 17 to 21, wherein the modified base of the 3′ end portion of the chimeric padlock probes is chosen from (i) an RNA base, (ii) a 2′-O-methoxy-ethyl base, (iii) a 2′-O-methyl RNA base, (iv) a 2′-fluoro base, (v) a DNA base or (vi) an LNA base.

23. The kit according to any one of claims 17 to 22, wherein the modified base at the 3′ end portion is positioned within at least two bases from the 3′ terminal base.

24. The kit according to any one of claims 17-23, wherein the padlock probe has any of the following designs:
(i) the 3′ terminal end base is a modified base and is positioned at the nucleotide position to be profiled of the RNA sample;
(ii) the 3′ terminal end base is a modified base, and the base next to the 3′ terminal end base is positioned at the nucleotide position to be profiled of the RNA sample; or
(iii) the base next to the 3′ terminal end base is a modified base and is positioned at the nucleotide position to be profiled of the RNA sample.

25. The kit according to any one of claims 17 to 24, wherein the 5′ terminal base of the 5′ end is phosphorylated or pre-adenylated.
